(19) United States
(12) Patent Application Publication
(10) Pub. No.: US 2019/0328300 A1
(43) Pub. Date: Oct. 31, 2019

(54) REAL-TIME ANNOTATION OF SYMPTOMS IN TELEMEDICINE

(71) Applicant: INTERNATIONAL BUSINESS MACHINES CORPORATION, Armonk, NY (US)

(72) Inventors: SEYEDBEHZAD BOZORGTABAR, Melbourne (AU); NOEL FAUX, Melbourne (AU); RAHIL GARNAVI, Melbourne (AU); SUMAN SEDAI, Melbourne (AU)

(21) Appl. No.: 15/964,542

(22) Filed: Apr. 27, 2018

(51) Int. Cl. A61B 5/16; H04N 7/14; H04L 29/06; G06F 17/27; G06K 9/00; G10L 15/26; G10L 25/66; G16H 80/00; A61B 5/00; A61B 5/11

(52) U.S. Cl. CPC A61B 5/165; A61B 2576/00; H04L 65/403; G06F 17/2785; G06K 9/00302; G06K 9/00335; G10L 15/265; G10L 25/66; G16H 80/00; A61B 5/7264; A61B 5/743; A61B 5/1116; A61B 5/1114; A61B 5/0077; A61B 5/1128; H04N 7/147

(57) ABSTRACT

A teleconferencing system includes a first terminal configured to acquire an audio signal and a video signal. A teleconferencing server in communication with the first terminal and a second terminal is configured to receive the video signal and the audio signal from the first terminal, in real-time, and transmit the video signal and the audio signal to the second terminal. A symptom recognition server in communication with the first terminal and the teleconferencing server is configured to receive the video signal and the audio signal from the first terminal, asynchronously, analyze the video signal and the audio signal to detect one or more indicia of illness, generate a diagnostic alert on detecting the one or more indicia of illness, and transmit the diagnostic alert to the teleconferencing server for display on the second terminal.

### BACKGROUND

The present invention relates to video conferencing and, more specifically, to a system for real-time annotation of facial, body, and speech symptoms in video conferencing.

Telemedicine is the practice by which healthcare can be provided with the healthcare practitioner and the patient being located in distinct locations, potentially over a great distance. Telemedicine creates an opportunity to provide quality healthcare to underserved populations and also to extend access to highly specialized providers. Telemedicine also has the potential to reduce healthcare costs.

### SUMMARY

A teleconferencing system includes a first terminal configured to acquire an audio signal and a video signal. A teleconferencing server in communication with the first terminal and a second terminal is configured to receive the video signal and the audio signal from the first terminal, in real-time and transmit the video signal and the audio signal to the second terminal. A symptom recognition server in communication with the first terminal and the teleconferencing server is configured to receive the video signal and the audio signal from the first terminal, asynchronously, analyze the video signal and the audio signal to detect one or more indicia of illness, generate a diagnostic alert on detecting the one or more indicia of illness, and transmit the diagnostic alert to the teleconferencing server for display on the second terminal.

A teleconferencing system includes a first terminal including a camera and a microphone configured to acquire an audio signal and a high-quality video signal and convert the acquired high-quality video signal into a low-quality video signal of a bitrate that is less than a bit rate of the high-quality video signal. A teleconferencing server in communication with the first terminal and a second terminal is configured to receive the low-quality video signal and the audio signal from the first terminal, in real-time, and transmit the low-quality video signal and the audio signal to the second terminal. A symptom recognition server in communication with the first terminal and the teleconferencing server is configured to receive the high-quality video signal and the audio signal from the first terminal, asynchronously, analyze the high-quality video signal and the audio signal to detect one or more indicia of illness, generate a diagnostic alert on detecting the one or more indicia of illness, and transmit the diagnostic alert to the teleconferencing server for display on the second terminal.

A method for teleconferencing includes acquiring an audio signal and a video signal from a first terminal. The video signal and the audio signal are transmitted to a teleconferencing server in communication with the first terminal and a second terminal. The video signal and the audio signal are transmitted to a symptom recognition server in communication with the first terminal and the teleconferencing server. Indicia of illness is detected from the video signal and the audio signal using multimodal recurrent neural networks. A diagnostic alert is generated for the detected indicia of illness. The video signal is annotated with the diagnostic alert. The annotated video signal is displayed on the second terminal.

A computer program product for detecting indicia of illness from image data, the computer program product including a computer readable storage medium having program instructions embodied therewith, the program instructions executable by a computer to cause the computer to acquire an audio signal and a video signal using the computer, detect a face from the video signal using the computer, extract action units from the detected face using the computer, detect landmarks from the detected face using the computer, track the detected landmarks using the computer, perform semantic feature extraction using the tracked landmarks, detect tone features from the audio signal using the computer, transcribe the audio signal to generate a transcription using the computer, perform natural language processing on the transcription using the computer, perform semantic analysis on the transcription using the computer, perform language structure extraction on the transcription, and use the multimodal recurrent neural networks to detect the indicia of illness from the detected face, extracted action units, tracked landmarks, extracted semantic features, tone features, the transcription, the results of the natural language processing, the results of the semantic analysis, and the results of the language structure extraction, using the computer.

### BRIEF DESCRIPTION OF THE SEVERAL VIEWS OF THE DRAWINGS

A more complete appreciation of the present invention and many of the attendant aspects thereof will be readily obtained as the same becomes better understood by reference to the following detailed description when considered in connection with the accompanying drawings, wherein:

FIG. 1 is a schematic illustrating a system for real-time annotation of facial symptoms in video conferencing in accordance with exemplary embodiments of the present invention;

FIG. 2 is a flow chart illustrating a manner of operation of the system illustrated in FIG. 1 in accordance with exemplary embodiments of the present invention;

FIGS. 3 and 4 include a process flow illustrating an approach for real-time annotation of facial symptoms in video conferencing in accordance with exemplary embodiments of the present invention;

FIG. 5 is a diagram illustrating a teleconference display in accordance with exemplary embodiments of the present invention; and

FIG. 6 shows an example of a computer system capable of implementing the method and apparatus according to embodiments of the present disclosure.

### DETAILED DESCRIPTION

In describing exemplary embodiments of the present invention illustrated in the drawings, specific terminology is employed for sake of clarity. However, the present invention is not intended to be limited to the illustrations or any specific terminology, and it is to be understood that each element includes all equivalents.

As discussed above, telemedicine creates an opportunity to extend healthcare access to patients who reside in regions that are not well served by healthcare providers. In particular, telemedicine may be used to administer healthcare to patients who might not otherwise have sufficient access to such medical services. However, there is a particular problem associated with remotely administering healthcare to patients; whereas a general practitioner may well be able to ask a patient to describe symptoms over a videoconference, health practitioners must often be able to recognize subtle symptoms from the manner in which the patient looks and acts.

Ideally, videoconferencing hardware used in telemedicine would be able to provide uncompressed super high definition video and crystal clear audio so that the health practitioner could readily pick up on minute symptoms, however, as there are significant practical limits to bandwidth, particularly at the patient's end as the patient may be located in a remote rural location, in an emerging country without built out high speed network access, or even at sea, in the air or in space, the quality of the audio and video received by the health provider may be inadequate and important but subtle symptoms may be missed.

Moreover, while it may be possible for high quality audio and video to be transmitted to the health provider asynchronously, as health care often involves natural conversation, the course of which is dependent upon the observations of the health provider, analyzing audio and video after-the-fact might not be an adequate means of providing health care.

Exemplary embodiments of the present invention provide a system for real-time video conferencing in which audio and video signals are acquired in great clarity and these signals are compressed and/or downscaled, to what is referred to herein as low-quality signals, for efficient real-time communication, while automatic symptom recognition is performed on the high-quality signals to automatically detect various subtle symptoms therefrom. The real-time teleconference using the low-quality signals is then annotated using the findings of the automatic symptom recognition so that the health care provider may be made aware of the findings in a timely manner to guide the health care consultation accordingly.

This may be implemented either by disposing the automatic symptom recognition hardware either at the location of the patient, or by sending the high-quality signals to the automatic symptom recognition hardware, asynchronously, as the real-time teleconference continues, and then superimposing alerts to the health care provider as they are determined.

The automatic symptom recognition hardware may utilize recurrent neural networks to identify symptoms in a manner described in greater detail below.

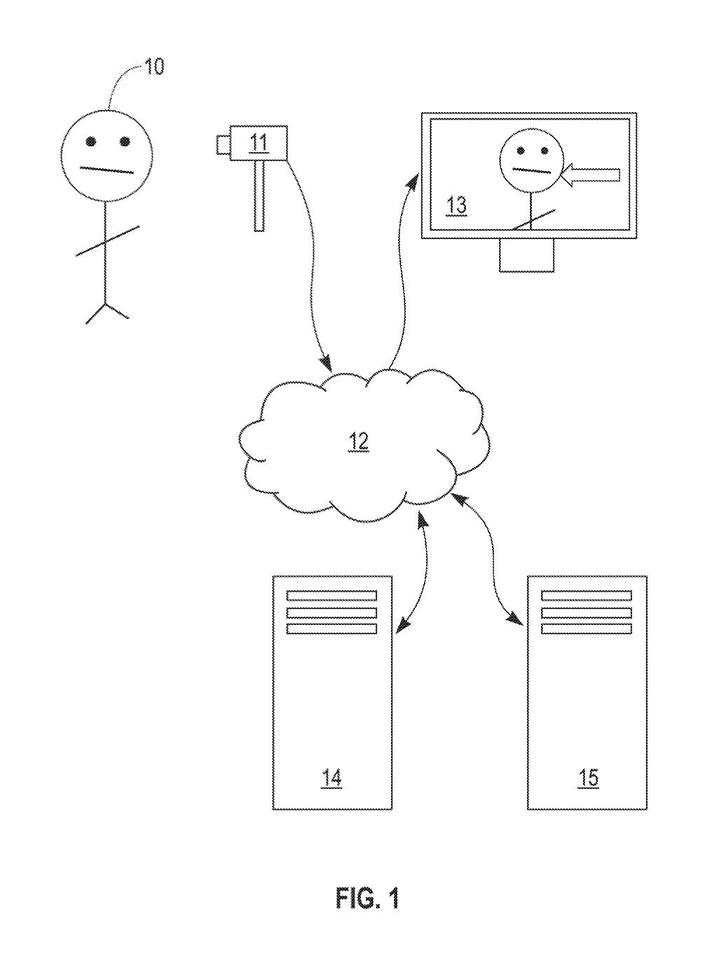

FIG. 1 is a schematic illustrating a system for real-time annotation of facial symptoms in video conferencing in accordance with exemplary embodiments of the present invention. A patient subject 10 may utilize a camera and microphone 11 and the sounds and appearance of the patient subject 10 may be acquired therefrom. Although element 11 is illustrated as a camera device, this depiction is merely an example, and the actual device may be instantiated as teleconferencing equipment, as a personal computer, or even as a mobile electronic device such as a smartphone or tablet computer including a camera/microphone. It is to be understood that the camera/microphone element 11 may additionally include analog-to-digital converters, a network interface, and a processor.

The camera/microphone 11 may digitize the acquired audio/video signal to produce high definition audio/video signals such as 4 k video conforming to an ultra-high definition (UHD) standard. The digitized signals may be in communication with a teleconferencing server 14, over a computer network 12, such as the Internet. The camera/microphone 11 may also reduce the size of the audio/video signals by down-scaling and/or utilizing a compression scheme such as H.264 or some other scheme. The extent of the reduction may be dictated by available bandwidth and various transmission conditions. The camera/microphone 11 may send the audio/video signals to the teleconferencing server 14 both as the high-quality acquired signal and as the scaled down/compressed signals, which may be referred to herein as the low quality signals. The high-quality signals may be sent asynchronously, for example, the data may be broken into packets which may reach the teleconferencing server 14 for processing upon complete transmission of some number of image frames, whereas the low-quality signals may be sent to the teleconferencing server 14 in real-time and the extent of the quality reduction may be dependent upon the nature of the connection through the computer network 12, while the high-quality signals may be sent without regard to connection quality.

The teleconferencing server 14 may perform two main functions, the first function may be to maintain the teleconference by relaying the low-quality signals to the provider terminal 13 in real-time. For example, the teleconferencing server 14 may receive the low-quality signal from the camera/microphone 11 and relay the low-quality signal to the provider terminal 13 with only a minimal delay such that a real-time teleconference may be achieved. The teleconferencing server 14 may also receive audio/video data from the provider terminal 13 and relay it back to the patient subject using reciprocal hardware at each end,

The second main function performed by the teleconferencing server 14 is to automatically detect symptoms from the high-quality signals, to generate diagnostic alerts therefrom, and to annotate the diagnostic alerts to the teleconference that uses the low quality signals. However, according to other approaches, the automatic detection and diagnostic alert generation may be handled by a distinct server, for example, a symptom recognition server 15. According to this approach, the camera/microphone 11 may send the high-quality signals, asynchronously, to the symptom recognition server 15 and send the low-quality signals, in real-time, to the teleconferencing server 14. The symptom recognition server 15 may then send the diagnostic alerts to the teleconferencing server 14 and the teleconferencing server 14 may annotate the teleconference accordingly.

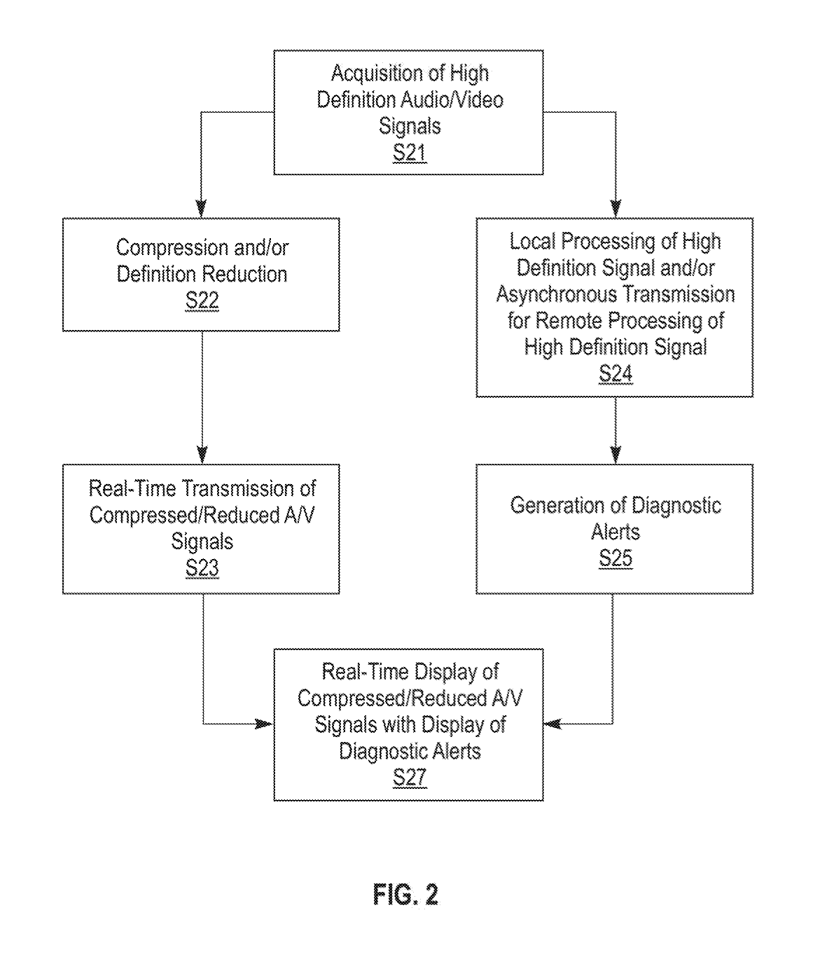

FIG. 2 is a flow chart illustrating a manner of operation of the system illustrated in FIG. 1 in accordance with exemplary embodiments of the present invention. As discussed above, first the telecommunications terminal of the patient subject may acquire the audio and video signals (Step S21). These high-quality signals may then either be processed locally or asynchronously transmitted to the symptom recognition server without reduction or lossy-type compression for processing (Step S24). Regardless of where processing is performed, the processing may result in the recognition of symptoms which may be used to generate diagnostic alerts (Step S25).

At substantially the same time, the low-quality signals may be transmitted to the teleconferencing server with a quality that is dependent upon the available bandwidth (Step S23). The teleconferencing server may receive the diagnostic alerts from the symptom recognition server and may annotate the diagnostic alerts thereon in a manner that is described in greater detail below (Step S27).

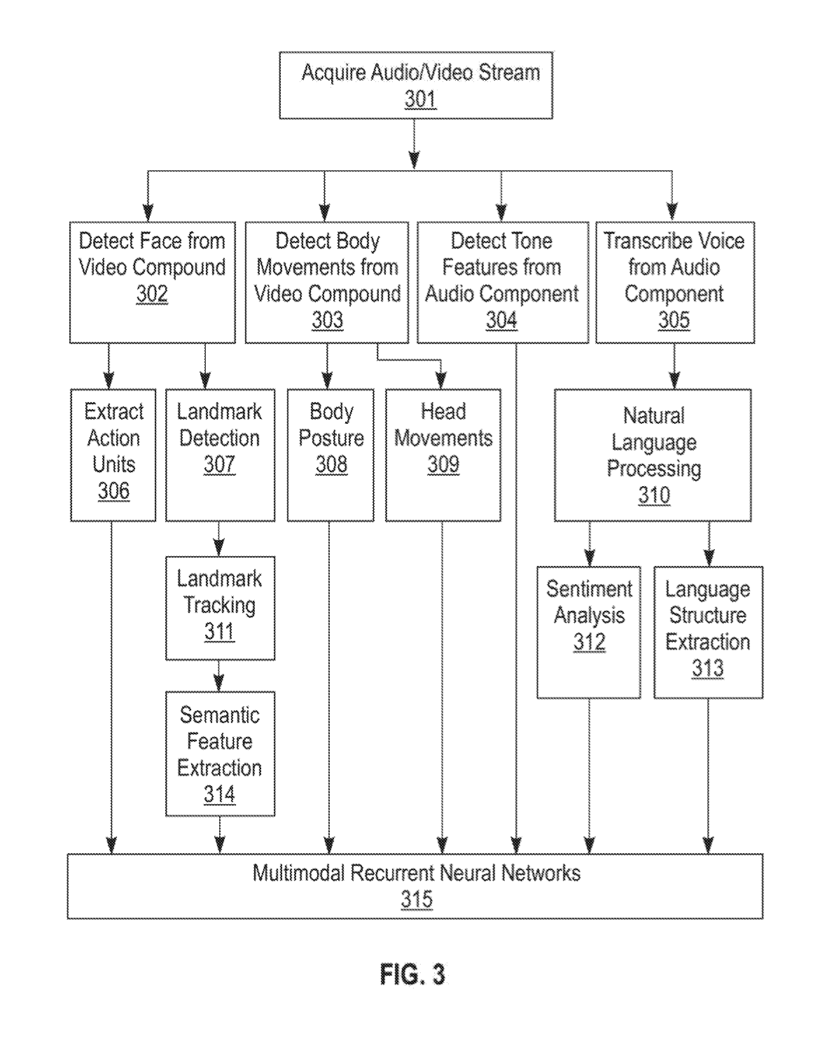

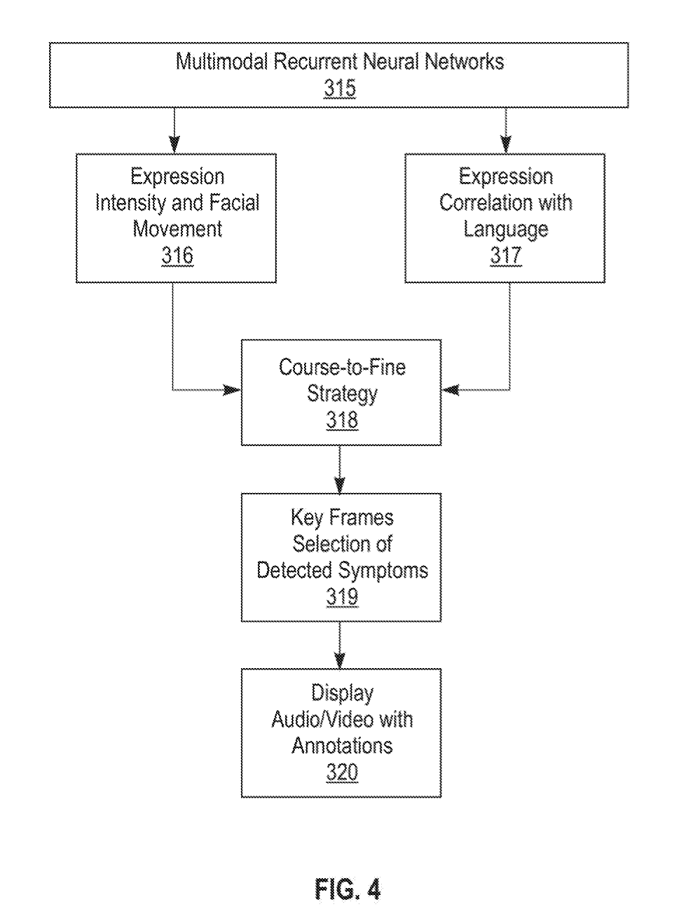

The symptom recognition server may utilize multimodal recurrent neural networks to generate the diagnostic alerts from the high-quality signals. FIGS. 3 and 4 illustrate an exemplary algorithm for performing this function.

As discussed above, high-definition audio and video signals may be acquired and sent asynchronously to the symptom recognition server (301). The symptom recognition server may thereafter use the video signal to perform facial detection (302) and to detect body movements (303). Thus, the video signal may include imagery of the patient subject's face and some component of the patient subject's body, such as neck, shoulders and torso. Meanwhile, from the audio signal, vocal tone may be detected (304) and language may be transcribed using speech-to-text processing (305).

From the detected face, action units may be extracted (306) and landmarks may be detected (307). Additionally, skin tone may be tracked to detect changes in skin tone. Action units, as defined herein, may include a recognized sequence of facial movements/expressions and/or the movement of particular facial muscle groups. In this step, the presence of one or more action units are identified from the detected face of the video component. This analysis may utilize an atlas of predetermined action units and a matching routine to match the known action units to the detected face of the video component.

While action unit detection may utilize facial landmarks, this is not necessarily the case. However, in either case, landmarks may be detected from the detected face (307). The identified landmarks may include points about the eyes, nose, chin, mouth, eyebrows, etc. Each landmark may be represented with a dot and the movement of each dot may be tracked from frame to frame (311). From the tracked dots, semantic feature extraction may be performed (314). Semantic features may be known patterns of facial movements, e.g. expressions and/or mannerisms, that may be identified from the landmark tracking.

Meanwhile, from the detected body movements (303), body posture (308) and head movements (309) may be determined and tracked. This may be accomplished, for example, by binarizing and then silhouetting the image data. Here body posture may include movements of the head, shoulders, and torso, together, while head movement may include the consideration of the movement of the head alone. Additionally, body posture may include consideration of arms and hands, for example, to detect subconscious displays of being upset or distraught such as interlacing stiffened fingers.

From the speech-to-text transcribed text (305), natural language processing may be performed (310. Natural language processing may be used to determine a contextual understanding of what the patient subject is saying and may be used to determine both the sentiment of what is being said (312), as well as the content of what is being said, as determined through language structure extraction (313).

The extracted action units (306), the semantic feature extraction (314), the body posture (308), the head movement (309), the detected tone (304), the sentiment analysis (312), and the language structure extraction (313) may all be sent to multimodal recurrent neural networks (315). The multimodal recurrent neural networks may use this data to determine an extent of expression of emotional intensity and facial movement (316) as well as an expression of correlation of features to language (317). The expression of emotional intensity and facial movement may represent a level of emotion displayed by the patient subject while the correlation of features to language may represent an extent to which a patient subject's non-verbal communication aligns with the content of what is being said. For example, discrepancy between facial/body movement and language/speech may be considered. These factors may be used to determine a probability of symptom display, as excessive emotional display may represent symptoms of health disorder and so might represent a deviation between features and language. However, exemplary embodiments of the present invention are not limited to using the multimodal recurrent neural networks to generate only these outputs, and any other features may be used by the multimodal recurrent neural networks to detect symptoms of health disorder, such as those features discussed above.

In assessing these characteristics, the expression of intensity and facial movement (316) may be compared to a threshold, and a value above the threshold may be considered a symptom. Moreover, the extent of correlation between expression and language (317) may similarly be compared to a threshold.

Here, the multi-output recurrent network may be used in modeling temporal dependencies of different feature modalities, where instead of simply aggregating video features over time, the hidden states of input features may be integrated by proposing addition layers to the recurrent neural network. In the network, there may be different labels for the training samples, which not only measure the facial expression intensity, but quantify the correlation between expression and language analytics. Especially, when there is a lack of expression in the patient face, but voice features may still be used to analyze the depth of emotion.

In assessing these and/or other outputs of the multi modal recurrent neural networks to detect symptoms of a health disorder, a course-to-fine strategy may be used (318) to identify potential symptoms within the audio/video signals. This information is used to identify key frames within the video where the potential symptoms are believed to be demonstrated. This step may be considered to be part of the diagnostic alert generation described above. These frames may be correlated between the flames of the high-quality signal and the low-quality signal and then the diagnostic alerts may be overplayed with the low-quality teleconference imagery, while in progress. While some amount of time may have passed between the time in which the symptoms were displayed and the time in which the diagnostic alert was generated, the diagnostic alert may be retrospective, and may include an indication that the diagnostic alert had been created, an indication of what facial features of the patient subject may have exhibited the symptoms, and also some way of replaying the associated video/audio as a picture-in-picture over the teleconference as it is progressing. The replay overlay may either be from the high-quality signal or the low-quality signal.

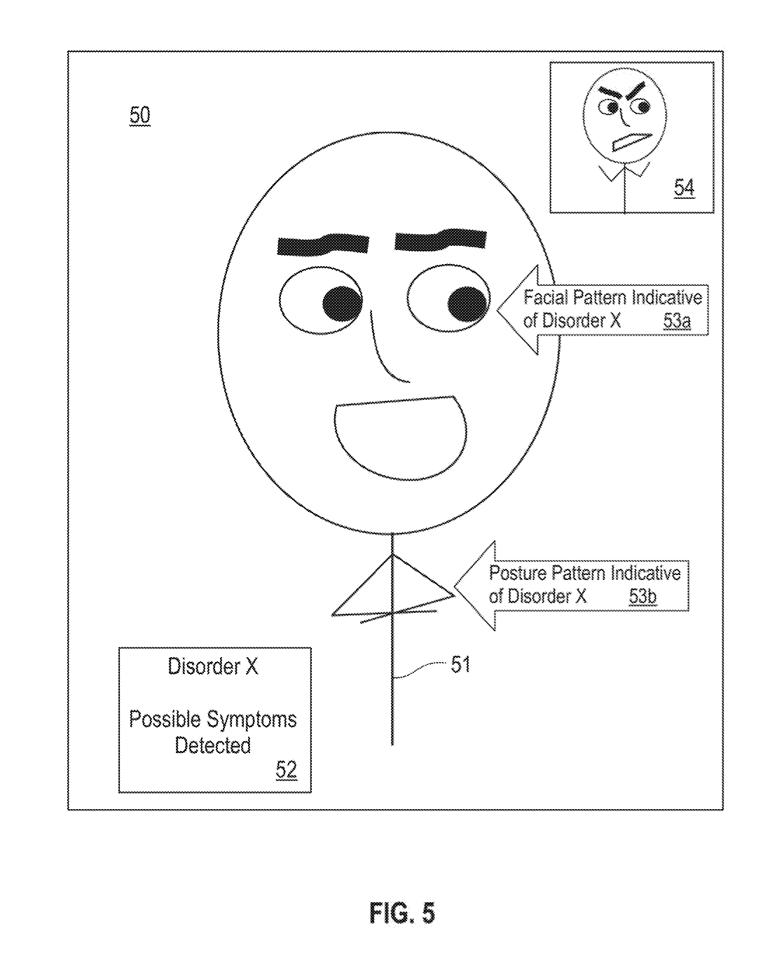

FIG. 5 is a diagram illustrating a teleconference display in accordance with exemplary embodiments of the present invention. The display screen 50 may include the real-time video image of the patient subject 51 from the low-quality signals. Diagnostic alerts may be overlaid thereon, including a textural alert 52 specifying the nature of the symptom detected, pointer alerts 53a and 53b referencing the detected symptoms and drawing attention to the areas of the patient subject responsible for displaying the symptoms, and/or a replay video box 54 in which a video clip around the key frame is displayed, for example, in a repeating loop.

Exemplary embodiments of the present invention need not perform symptom recognition on a high-quality video signal. According to some exemplary embodiments of the present invention, the camera/microphone may send the low-quality video signal to the symptom recognition server and the symptom recognition server may either perform analytics on the low-quality video signal by performing a less sensitive analysis or the symptom recognition server may up-sample the low-quality video signal to generate an enhanced-quality video signal from the low quality video signal, and then symptom recognition may be performed on the enhanced-quality video signal.

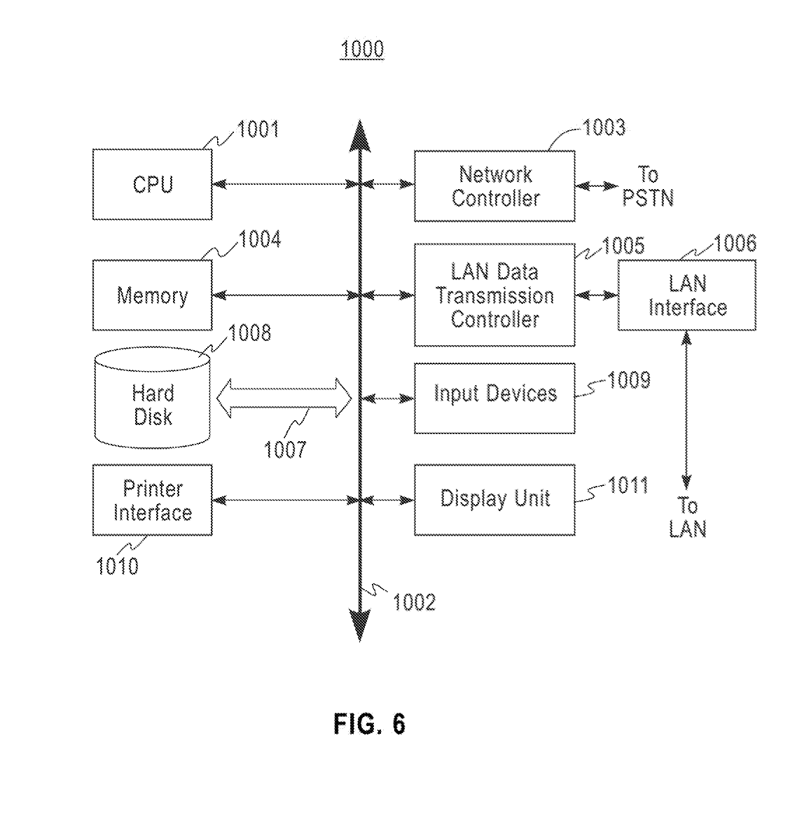

FIG. 6 shows another example of a system in accordance with some embodiments of the present invention. By way of overview, some embodiments of the present invention may be implemented in the form of a software application running on one or more (e.g., a “cloud” of) computer system(s), for example, mainframe(s), personal computer(s) (PC), handheld computer(s), client(s) server(s), peer-devices, etc. The software application may be implemented as computer readable/executable instructions stored on a computer readable storage media (discussed in more detail below) that is locally accessible by the computer system and/or remotely accessible via a hard wired or wireless connection to a network, for example, a local area network, or the Internet.

Referring now to FIG. 6, a computer system (referred to generally as system 1000) may include, for example, a processor e.g., central processing unit (CPU) 1001, memory 1004 such as a random access memory (RAM), a printer interface 1010, a display unit 1011, a local area network (LAN) data transmission controller 1005, which is operably coupled to a LAN interface 1006 which can be further coupled to a LAN, a network controller 1003 that may provide for communication with a Public Switched Telephone Network (PSTN), one or more input devices 1009, for example, a keyboard, mouse etc., and a bus 1002 for operably connecting various subsystems/components. As shown, the system 1000 may also be connected via a link 1007 to a non-volatile data store, for example, hard disk, 1008.

In some embodiments, a software application is stored in memory 1004 that when executed by CPU 1001, causes the system to perform a computer-implemented method in accordance with some embodiments of the present invention, e.g., one or more features of the methods, described with reference to FIGS. 4 and 5.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised. structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Exemplary embodiments described herein are illustrative, and many variations can be introduced without departing from the spirit of the invention or from the scope of the appended claims. For example, elements and/or features of different exemplary embodiments may be combined with each other and/or substituted for each other within the scope of this invention and appended claims.

1. A teleconferencing system, comprising:
a first terminal including a camera and a microphone configured to acquire an audio signal and a high-quality: video signal and convert the acquired high-quality video signal into a low-quality video signal of a Nitrate that is less than a hit rate of the high-quality video signal;
a teleconferencing server in communication with the first terminal and a second terminal and configured to receive the low-quality video signal and the audio signal from the first terminal, in real-time and transmit the low-quality video signal and the audio signal to the second terminal; and
a symptom recognition server in communication with the first terminal and the teleconferencing server and configured to receive the high-quality video signal and the audio signal from the first terminal, asynchronously, analyze the high-quality video signal and the audio signal to detect one or more indicia of illness, generate a diagnostic alert on detecting the one or more indicia of illness, and transmit the diagnostic alert to the teleconferencing server for display on the second terminal.

2. The system of claim 1, wherein the symptom recognition server is configured to detect the indicia of illness from the high-quality video signal and the audio signal using multimodal recurrent neural networks.

3. The system of claim 2, wherein the symptom recognition server is configured to detect the indicia of illness from the high-quality video signal by:
detecting a face from the high-quality video signal;
extracting action units from the detected face;
detecting landmarks from the detected face;
tracking the detected landmarks;
performing semantic feature extraction using the tracked landmarks; and
using the multimodal recurrent neural networks to detect the indicia of illness from the detected face, extracted action units, tracked landmarks, and extracted semantic features.

4. The system of claim 2, wherein the symptom recognition server is configured to detect the indicia of illness from the high-quality video signal by:
detecting a body posture from the high-quality video signal;
tracking head movements from the high-quality video signal; and
using the multimodal recurrent neural networks to detect the indicia of illness from the detected body posture and tracked head movements.

5. The system of claim 2, wherein the symptom recognition server is configured to detect the indicia of illness from the audio signal by:
detecting tone features from the audio signal;
transcribing the audio signal to generate a transcription;
performing natural language processing on the transcription;
performing semantic analysis on the transcription;
performing language structure extraction on the transcription; and
using the recurrent neural networks to detect the indicia of illness from the detected tone features, the transcription, the results of the natural language processing, the results of the semantic analysis, and the results of the language structure extraction.

6. The system of claim 1, wherein the first terminal is configured to convert the video signal into a low-quality video signal of less bitrate by reducing a resolution of the high-quality signal, by reducing a framerate of the high-quality signal, or by compressing the high-quality signal.

7. The system of claim 1, wherein the symptom recognition server is part of or locally connected to the first terminal.

8. The system of claim 1, wherein the teleconferencing server is in communication with the first terminal and the second terminal over the Internet or another wide-area network.

9. The system of claim 1, wherein the second terminal is configured to display the low-quality video signal as part of a teleconference and the teleconferencing server is configured to overlay the diagnostic alert on the display of the second terminal.

10. The system of claim 9, wherein the teleconferencing server is configured to overlay the diagnostic alert on the display of the second terminal in the form of a textual alert.

11. The system of claim 9, wherein the teleconferencing server is configured to overlay the diagnostic alert on the display of the second terminal in the form of a graphic element that highlights or emphasizes a part of a face or body that the indicia of illness are based on.

12. The system of claim 9, wherein the teleconferencing server is configured to overlay the diagnostic alert on the display of the second terminal in the form of an annotation, highlighting, or other marking on a textual transcription of the audio signal.

13. The system of claim 9, wherein the teleconferencing server is configured to overlay the diagnostic alert on the display of the second terminal in the form of a picture-in-picture element that includes a replaying of a portion of the high-quality video signal that the indicia of illness are based on.

14. A method for teleconferencing, comprising:
acquiring an audio signal and a video signal from a first terminal;
transmitting the video signal and the audio signal to a teleconferencing server in communication with the first terminal and a second terminal;
transmitting the video signal and the audio signal to a symptom recognition server in communication with the first terminal and the teleconferencing server;
detecting indicia of illness from the video signal and the audio signal using multimodal recurrent neural networks;
generating a diagnostic alert for the detected indicia illness;
annotating the video signal with the diagnostic alert; and
displaying the annotated video signal on the second terminal.

15. The method of claim 14, wherein detecting the indicia of illness from the video signal comprises:
detecting a face from the video signal;
extracting action units from the detected face;
detecting landmarks from the detected face;
tracking the detected landmarks;
performing semantic feature extraction using the tracked landmarks; and
using the multimodal recurrent neural networks to detect the indicia of illness from the detected face, extracted action units, tracked landmarks, and extracted semantic features.

16. The method of claim 14, wherein detecting the indicia of illness from the video signal comprises:
detecting a body posture from the video signal;
tracking head movements from the video signal; and
using the multimodal recurrent neural networks to detect the indicia of illness from the detected body posture and tracked head movements.

17. The method of claim 14, wherein detecting the indicia of illness from the audio signal comprises;
detecting tone features from the audio signal;
transcribing the audio signal to generate a transcription;
performing natural language processing on the transcription;
performing semantic analysis on the transcription;
performing language structure extraction on the transcription; and
using the recurrent neural networks to detect the indicia of illness from the detected tone features, the transcription, the results of the natural language processing, the results of the semantic analysis, and the results of the language structure extraction.

18. The method of claim 14, wherein a bit rate of the video signal is reduced prior to transmitting the video signal to the symptom recognition server.

19. The method of claim 14, wherein the video signal is up-sampled prior to detecting the indicia of illness from the video signal.

20. A computer program product for detecting indicia of illness from image data, the computer program product comprising a computer readable storage medium having program instructions embodied therewith, the program instructions executable by a computer to cause the computer to:
acquiring an audio signal and a video signal using the computer;
detecting a face from the video signal using the computer;
extracting action units from the detected face using the computer;
detecting landmarks from the detected face using the computer;
tracking the detected landmarks using the computer;
performing semantic feature extraction using the tracked landmarks;
detecting tone features from the audio signal using the computer;
transcribing the audio signal to generate a transcription using the computer;
performing natural language processing on the transcription using the computer;
performing semantic analysis on the transcription using the computer;
performing language structure extraction on the transcription; and
using the multimodal recurrent neural networks to detect the indicia of illness from the detected face, extracted action units, tracked landmarks, extracted semantic features, tone features, the transcription, the results of the natural language processing, the results of the semantic analysis, and the results of the language structure extraction, using the computer,

21. The computer program product of claim 20, wherein the program instructions executable by a computer further causes the computer to detect the indicia of illness from the video signal by:
detecting a body posture from the video signal, using the computer;
tracking head movements from the video signal, using the computer; and
using the multimodal recurrent neural networks to detect the indicia of illness from the detected body posture and tracked head movements, using the computer.

22. The computer program product of claim 20, wherein the program instructions executable by a computer further causes the computer to generate a diagnostic alert when the multimodal recurrent neural networks detect the indicia of illness.
